(19) United States
(12) Patent Application Publication
(10) Pub. No.: US 2006/0281143 A1
(43) Pub. Date: Dec. 14, 2006

(54) Method and apparatus for automatic cell and biological sample preparation and detection

(75) Inventors: Benjamin Y.H. Liu, North Oaks, MN (US); Friedrich Srienc, Lake Elmo, MN (US); Christian Lavarreda, Minneapolis, MN (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: MSP Corporation, Shoreview, MN (US)

(21) Appl. No.: 11/387,453

(22) Filed: Mar. 23, 2006

Related U.S. Application Data
(60) Provisional application No. 60/667,280, filed on Apr. 1, 2005.

(51) Int. Cl. C12Q 1/04; B01J 19/00; C12M 1/34

(52) U.S. Cl. 435/34; 422/102; 422/130; 435/287.3; 435/286.5; 435/297.2

(57) ABSTRACT

A system identifies or screens one or more biological materials. The system includes at least one bioreactor adapted to provide biological samples and an automated sample handler comprising at least one bi-directional pump to transport biological samples, reagents and fluids, at least one reaction chamber with at least two regions separated by a semi-permeable membrane and fitted with a thermoelectric module for cooling and heating the chamber. At least one multi-positional valve directs transport of the biological samples, reagents and fluids into and out of the reaction chamber and a micro-processor to control operations of the sample handler. A detector is used to identify the biological material in the samples and a sample repository collects the identified biological samples.

### CROSS-REFERENCE TO RELATED APPLICATION

The present application is based on and claims the benefit of U.S. provisional patent application Ser. No. 60/667,280, filed Apr. 1, 2005, the content of which is hereby incorporated by reference in its entirety.

### FIELD OF THE INVENTION

This invention relates to a method and apparatus for preparing cell and biological samples suspended in a fluid (liquid) for detection by a suitable detector.

### BACKGROUND OF THE INVENTION

Cell populations are inherently heterogeneous. The cell-to-cell variability can be attributed (i) to genetic differences, (ii) to different cell cycle stages of individual cells, (iii) to different microenvironments individual cells are exposed to, and (iv) to stochastic variations caused by the small number of molecules that regulate certain cellular events. Assessing cellular heterogeneity is important in many areas including clinical research and diagnostics, in which it is critical to recognize, for instance, aberrant cells such as cancer cells. It is also important in biotechnology, where cells are cultivated in cultures to produce valuable products. It is clear that in such cultures, cells that are not productive are not desirable, while cells that are extremely productive are highly desirable. Cellular heterogeneity can be detected with an analysis approach that operates at the single cell level such as microscopy or flow cytometry.

Flow cytometry has proven to be an invaluable tool in clinical, industrial, and research settings. However, one of its limitations is the extensive amount of sample handling and processing that is required. While many published articles describe the development of core FCM instrumentation, there exist rather few studies focusing on the automation of flow cytometry processes. The original designs involved the construction of sample chambers to allow for the on-line addition of reagents and the reduction of the sample transit time. Omann G M, Coppersmith W, Finney D A, Sklar L A. A convenient on-line device for reagent addition, sample mixing, and temperature control of cell suspensions in flow cytometry, Cytometry 1985; 6:69-73; Pennings A, Speth P, Wessels H, Haanen C. Improved flow cytometry of cellular DNA and RNA by on-line reagent addition, Cytometry 1987; 8:335-338; Kelley K A. Sample station modification providing on-line reagent addition and reduced sample transit time for flow cytometers, Cytometry 1989; 10:796-800; and Lindberg W, Ruzicka J, Christian G D. Flow injection cytometry: a new approach for sample and solution handling inflow cytometry, Cytometry 1993; 14:230-236.

These devices standardized time dependent staining procedures, and increased the temporal resolution of kinetic samples so that events could be detected seconds after reagent addition. More recent advances have focused on developing systems capable of analyzing a large number of samples in a short period of time. Such advances extend the utility of flow cytometry to drug design and other applications requiring high throughput screening. Edwards BS, Kuckuck F, Sklar L A. Plugflow cytometry: an automated coupling device for rapid sequential flow cytometric sample analysis, Cytometry 1999; 37:156-159; Edwards B S, Kuckuck F, Prossnitz E R, Okun A, Ransom J T, Sklar L A. Plug flow cytometry extends analytical capabilities in cell adhesion and receptor pharmacology, Cytometry 2001; 43:211-216; and Kuckuck F, Edwards B S, Sklar L A. High throughput flow cytometry, Cytometry 2001; 44:83-90.

In biotechnology, growing cell cultures are conventionally characterized by their overall bulk properties by monitoring changes in substrate, product, and byproduct concentrations in the extra-cellular environment. However, it is well known that a cell population is the sum of individual cells, all of which contribute differently to the overall observed behavior. Therefore, an accurate description of this heterogeneity is of paramount interest as it provides a basis for the implementation of more efficient control strategies for bioprocesses. Mantzaris NV, Srienc F, Daoutidis P. Nonlinear productivity control using a multi-staged cell population balance model, Chemical Engineering Science 2002; 57:1-14.

Flow cytometry has been used in many basic and applied studies. For instance, it has been used to describe the heterogeneity in substrate uptake rates (Natarajan A, Srienc F. Dynamics of glucose uptake by single Escheirichia coli cells, Metabolic Engineering 1999; 1:320-333) and in intracellular protein accumulation yielding information about cell populations that otherwise remains hidden. Kromenaker S J, Srienc F. Cell-cycle dependent protein accumulation by producer and non-producer murine hybridoma cell lines: a population analysis, Biotechnology and Bioengineering 1991; 38:665-677. Additionally, it facilitated the recent explosion in cell cycle research that has occurred over the past decade and that was critical for determining the mechanistic details of the regulatory network that plays a major role in controlling relevant cellular processes. Moreover, changes in cell cycle distributions observed using flow cytometry data have been shown to be directly correlated to cell growth rates and can be used to predict future trends in growth. Kromenaker S J, Srienc F. Cell-cycle dependent protein accumulation by producer and non-producer murine hybridoma cell lines: a population analysis, Biotechnology and Bioengineering 1991; 38:665-677; Leelavatcharamas V., Emery A. N., Al-Rubeai M. Monitoring the proliferative capacity of cultured animal cells by cell cycle analysis, 1996; 1-15.

Although flow cytometry is an ideal tool to accurately describe these phenomena, there is an extensive amount of sample preparation required to quantify and analyze changes in cellular properties that occur during a culture process. Thus, it is desirable to automate the sampling and sample preparation procedures that are necessary for analysis. While the devices mentioned above are very innovative and useful, they are limited to performing simple staining reactions that require a single step.

### SUMMARY OF THE INVENTION

The present invention includes a system to identify or screen one or more biological materials comprising at least one bioreactor adapted to provide biological samples containing cells, cell components, cell-drive materials or a combination thereof in a fluid and an automated sample handler that comprises at least one bi-directional pump to transport biological samples, radiations and fluids. The sample handler includes at least one reaction chamber with at least two regions separated by a semi-permeable membrane and fitted with a thermoelectric module for cooling and heating the chamber and at least one multi-positional valve to direct transport of the biological samples, reagents and fluids into and out of the reaction chamber and a micro-processor to control operations of the sample handler. A detector is used to identify the biological material in the samples and the sample repository collects identified biological samples.

The present invention also comprises an apparatus for preparing a biological sample for detection, the apparatus comprises a chamber with a semi-permeable membrane, a mechanism to transfer fluid including a pump, a valving mechanism in cooperation with the mechanism to transfer fluid for directing flow of fluid into the chamber, and at least one of the following two components: (1) a thermoelectric module for cooling and heating the chamber or (2) an microwave mechanism to irradiate the biological sample within the chamber.

The present invention also comprises an apparatus for preparing a biological sample for detection, the apparatus comprising a chamber with a semi-permeable membrane, a mechanism to transfer fluid including a pump, a valving mechanism in cooperation with the mechanism to transfer fluid for directing flow of fluid into the chamber, the pump being one of the following three types: (1) a multi-cylinder piston pump, (2) a bi-directional multi-cylinder piston pump or (3) a multi-cylinder piston pump operated with a stepper motor. The chamber may also include a magnetic stirrer.

The present invention also comprises an apparatus for preparing a biological sample for subsequent processing by a detection mechanism. This apparatus comprises a housing with an inlet and an outlet, a chamber for preparing a biological sample and has a mechanism to provide motive force to transport the biological sample into the inlet, a biological sample preparation mechanism that prepares the biological sample, and a biological sample delivery mechanism that transports the biological sample through the outlet. The apparatus may further include an input receptacle for providing a biological sample and the input receptacle being in fluid communication with the inlet such that the biological sample is transportable through the inlet and further including an outlet receptacle for storing the prepared biological samples. The apparatus may further include a plurality of input receptacles and a plurality of output receptacles and an input valving mechanism for providing fluid transport passages such that the biological samples may be drawn from selected input receptacles. An outlet valving mechanism provides transport passages to prepared biological samples for transport to selected output receptacles. The apparatus may also include a well plate having a plurality of wells a portion of which include biological samples for transport through the inlet and a portion of which are for storing prepared biological samples received through the outlet. The apparatus may also include a platform operated by stepper motors and being moveable horizontally in two perpendicular directions such that a portion of the plurality of wells are positioned in fluid communication with the inlet for drawing biological samples. This apparatus may also include a portion of the wells being positioned by movement of the platform to a position for receiving prepared biological samples. Additionally, the apparatus may include a biological material source in which biological material is developed. The biological material source is in transport communication with the inlet for providing biological material to the inlet. A flow cytometer is in fluid communication with the outlet and receives and detects prepared biological material from the outlet.

The present invention also includes a biological sample preparation and detection apparatus for preparing and detecting biological factors within a biological sample, the apparatus includes a housing having a chamber for receiving the biological sample and an inlet in fluid communication with the chamber. A transport mechanism transports the biological sample through the inlet, and a processing mechanism prepares the biological sample for subsequent detection of selected biological factors. A detection mechanism detects the selected biological factors from the prepared biological material. A thermoelectric cooler and heater in heat transfer relationship with the temperature provides a selected temperature environment within the chamber. The detection mechanism of this apparatus may also include a light source capable of inducing fluorescent light emission from the biological material, a first photo detector and a first filter positioned to permit fluorescent light to reach the photo detector. The apparatus may include at least one additional photo detector and at least one additional filter to detect fluorescent light in wave length ranges different than the first photo detector and the first filter. The apparatus may also include a biological material source for developing biological material with the source being in fluid communication with the inlet. A valving mechanism produces a fluid passage for transport of biological material to the inlet and produces fluid passages for cleaning fluid to flow to cleaning passages between the biological material source and the inlet.

The present invention also includes a process to identify or screen a biological material and the fluid sample comprising removing a fluid sample from a cell suspension by pulling the fluid sample into a sample loop with a bi-directional pump that is downstream from the sample loop. Transporting the sample in the sample loop to reaction chamber having at least two regions separated by a semi-permeable membrane by pushing the sample from the sample loop to the reaction chamber with the bi-directional pump. Contacting the sample in the reaction chamber with at least one reagent to provide a detectable biological material and transporting the detectable biological material to a detector. Identifying the detectable biological material with the detector to provide an identified biological material and transporting the identified biological material to a repository.

The present invention also comprises a method for transporting a sample of biological material from a first location to a second location through a fluid passage. The method includes providing a valving mechanism between the first location and the second location the valving mechanism having a plurality of ports to effect different fluid passages. A repository is provided, the repository being in fluid communication with one port of the valve and a pump is provided in fluid communication with the repository. The pump is operated in a suction mode when the valve produces a first passage between the first location and the repository such that the sample is positioned within the repository and operating the pump in a pumping mode when the valve produces a second passage between the repository and the second location such that the sample of biological material is transported from the repository to the second location.

The present invention also includes an apparatus for transporting a sample of biological material. The apparatus comprises a source of biological material and a destination for receiving the biological material. A valving mechanism has a plurality of ports to effect different fluid passages and is in fluid communication with the source and the destination. A repository temporarily receives a sample of the biological material, and a pump in fluid communication with the repository acts on the repository in suction mode and in pumping mode. When the valve is operated to produce a first passage between the source and the receptacle, the pump is operational in a suction mode and draws a sample of the biological material into the repository. When the valving mechanism is in a second position producing a passage between the repository and the destination, the pump is operational in a pumping mode to transport the biological sample to the destination.

### BRIEF DESCRIPTION OF THE FIGURES

FIG. 1A is a schematic view of a micro-chamber reactor with temperature control, fluid stirring, and microwave exposure.

FIG. 1B is a sectional view taken along the line 1B-1B in FIG. 1A.

FIG. 2 is a schematic view of a system for automatic fluid handling and transfer for the micro-chamber reactor.

FIG. 3A is a schematic view of a micro-chamber reactor with optical system for cell detection and counting.

FIG. 3B is a sectional view taken along the line 3B-3B in FIG. 3A.

FIG. 4 is a schematic view of a fluid handling system for sampling cells from multiple sources and measurement by a flow cytometer.

FIG. 5 is a schematic view of a manual sample introduction into a sample well and manual removal of treated sample in a sample receiving well

FIG. 6 is a schematic view of an automatic sample extraction from a bio-reactor with automatic sample line flushing and cleaning.

FIG. 7 is a schematic view of a fluid transfer system using coiled tubing to eliminate cell transport through the pumping chamber of a positive displacement pump

FIG. 8 is a schematic view of a multi-chamber micro-reactor for differential cell analysis following treatment

FIG. 9A is a schematic view of a 96-well plate mounted on an XY stage for automatic withdrawal of 48 cell samples from the wells and the delivery of treated samples into the remaining 48 empty wells following cell treatment by the automatic cell preparation and staining system.

FIG. 9B is a top schematic view of a 96-well plate.

### DETAILED DESCRIPTION OF THE INVENTION

The present invention includes an apparatus and method that makes it possible to carry out automatic sample preparation of cells and other biological materials for detection by flow cytometry. Presently such techniques are largely manual. The technique of this invention is combined with an optical detector to form an automatic flow cytometer, in which sample preparation and cell detection are both carried out automatically in the same instrument without human intervention. The method and apparatus are useful not only for detecting biological cells, but also for proteins, peptides, and other biological material suspended in a fluid.

Like reference characters will be used throughout the drawings to indicate like elements.

For purposes of the present invention the following terms are defined as:

Biological sample means a fluid sample containing suspended cells, cellular material, fungi, antigens, biological factors, immunogens, proteins, and other material of biological origin generally having a size substantially larger than the size of the molecules that comprise the fluid.

Fluid means a liquid. The liquid may be a pure liquid or a solution. The fluid may also be a liquid containing suspended biological material in which case it is also referred to as a biological sample.

Biological factors mean compounds made by living organisms or attached to living organisms that have biological or physiological activities. Biological factors include but are not limited to biological markers, antibodies, cytokines, growth factors, and other peptides, proteins, lipids and carbohydrates produced biologically.

Semi-permeable membrane means a membrane material that retains on one side selected biological material while permitting fluid molecules and other nonselected material to permeate therethrough;

Cytometer as used herein includes an instrument that not only counts cells but other biological material as defined herein. For purposes of convenience occasionally only the word “cell” is used in this application but should be understood to also mean other biological material as defined herein.

Housing means a structural framework on which various components of the present invention are mounted. The housing may have an enclosure for decorative or protective purposes or it may be a simple structural framework without a cover.

A multi well plate includes a plate that is substantially flat with sufficient thickness to contain a multitude of wells, i.e. receptacles. The wells are usually arranged in parallel, rows and columns. The wells are usually quite small with a volume typically in the range from a fraction of a milliliter to a few milliliters.

The present invention includes a system that is capable of many individual steps including automated sampling, fixing, staining, washing, diluting and analyzing cell and biological samples.

Typical variables that are of interest in a bioprocess setting are cell counts, cell viability, and the population distribution of cells and cellular components. By monitoring these variables over time, these data can be used to construct detailed kinetic pictures of the cell populations, which would allow for a deeper understanding of the kinetics of these processes. This invention includes several improvements to a previously described device (Zhao R, Natarajan A, Srienc F. A flow injection flow cytometry system for on-line monitoring of bioreactors, Biotechnology and Bioengineering 1999; 62:609-617.3) that enables the determination of cell counts in addition to analysis of cell property distributions.

These improvements include a positive displacement pump operated by a stepper motor to inject a known volume of fluid into the micro-chamber reactor such that the total number of events detected by the flow cytometer yields cell count data. In addition, a coil of tubing is used for sample transfer to greatly reduce cell damage during transfer. The system is also interfaced with a flow cytometer or combined with a built-in cell detector with fixed optical path settings that can operate without daily adjustments allowing the apparatus to function over extended periods of time with minimal user intervention. To demonstrate the utility of the system, yeast cultures expressing Green Fluorescent Protein (GFP) were monitored automatically for over two days while detailed kinetic information on the culture was obtained. Such information could be obtained only with significant effort using conventional methods.

Cell and biological sample preparation is conventionally carried out manually in many steps. In these steps reagents are added to cell suspensions and the cells are exposed to these reagents at defined temperatures and for specific amounts of time. During this incubation time the reagents react with the cells to make them permeable, to make them mechanically resistant to further treatment, or to attach fluorescent molecules to specific targets in the cells. After each treatment with a reagent the reagent has to be separated from the cells. This is done typically by centrifugation which sediments cells and allows for the reagent in the supernatant to be removed. Cells are often resuspended in a washing solution and the centrifugation and cell separation steps are repeated. Complex cell staining operations can involve several incubation steps with different reagents at different temperatures with washing steps in-between. A precise temperature in these incubation steps is important because the reaction rates of the staining reactions are usually strongly dependent on temperature. Steady temperatures are therefore a prerequisite for highly reproducible results. Of equal importance is that the cell environment is homogeneous, which is ensured by careful mixing of the cell suspensions.

In the present invention, biological cells grown in a culture and suspended in the growth medium are separated from the suspending fluid (liquid) by passing the cell sample into a micro-chamber reactor and then through an outlet separated by a semi-permeable membrane. Washing and reagent fluids are then pumped into the chamber and through the membrane to prepare the sample for detection by a flow cytometer. The preparation steps include washing, fixing, staining, incubating and diluting. The stained cells are then delivered by a mobile phase fluid to a flow cytometer for detection and counting.

The micro-chamber reactor is provided with a magnetic stirrer and a Peltier heating and cooling module to provide a controlled temperature environment in which to carry out the sample preparation steps. This greatly improves the accuracy and precision of the sample preparation process. A microwave device is also included permitting the use of accelerated, microwave assisted cell preparation techniques.

The fluid transfer system used includes a bidirectional variable-speed positive displacement pump with a stepper motor drive mechanism to pump the cell sample into the micro-chamber reactor and circulate the wash and reagent fluids through the chamber. The pump is operated at a low speed to minimize cell damage when cell samples are being transferred, and at a high speed when wash fluid is circulated to reduce the time needed for washing. Yet other speeds can be used when cells are undergoing fixing and staining in order to minimize the amount of reagent fluids used. The bidirectional positive displacement stepper-motor driven pump is also used to precisely deliver known volumes of cell suspension into the micro-chamber to enable the accurate generation of cell concentration data based on cell counts generated by a high accuracy flow cytometric detector.

An automatic flow cytometer technique is described in which the automated cell sample preparation technique is combined with a cell detector based on optical sensing to achieve a truly automatic cell measuring system, from sample preparation to automated cell detection and counting. The optical detector used includes a sensor to detect elastically scattered light from the cells and one or more sensors to detector light produced by laser induced fluorescence.

Additional aspects of the invention are the use of a multiplexed sampling technique in which a micro-chamber reactor is used to extract samples from two or more bioreactors for automated reactor monitoring, and the use of a multi-chamber reactor for differential cell analysis needed for research and drug development. A further aspect of the invention is to use a coil of tubing to collect and deliver a cell sample by a mobile phase fluid to greatly reduce or totally eliminate cell damage during transport, and combining the sample preparation system with a multi-well plate for cell screening or drug development applications.

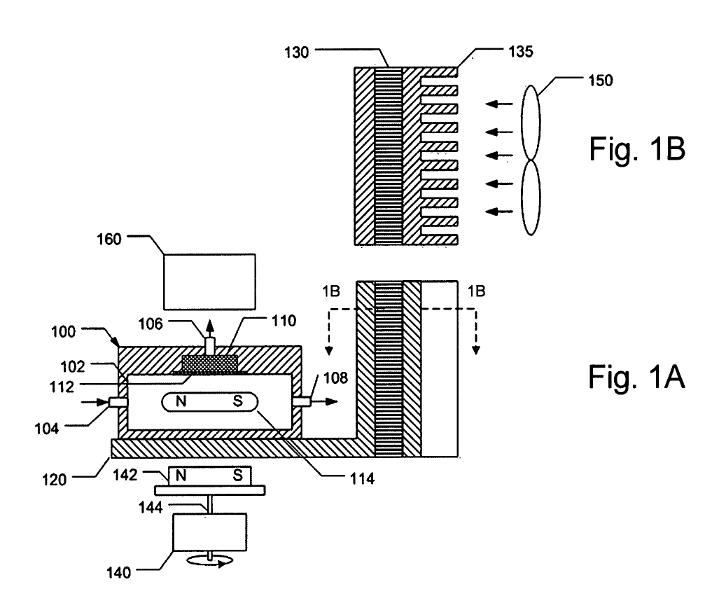

FIG. 1A shows a schematic diagram of the micro-chamber reactor 100, with an interior volume 102 and multiple ports to allow fluid (liquid) to enter and exit this interior chamber. For micro-analytical purposes, the chamber is usually quite small, typically a few hundred microliter (μl) in volume. For other applications, larger chambers can be used. However, the operational steps involved would remain substantially the same irrespective of the chamber size.

Port 104 is an entry port, through which fluids are let into the chamber. Fluids entering through this port include cell and biological samples, fixatives and stains, and the mobile-phase fluid for washing and for diluting the cells and for transporting the stained cells to the flow cytometer for counting. Following cell treatment, sterilizing and cleaning fluid can also be introduced through this port to prevent cross contamination when multiple cell samples are prepared for analysis using the same device.

Ports 106 and 108 are exit ports. Inside the chamber is a semi-permeable membrane 112 with a pore size smaller than the size of the cells or other biological material to be treated. When fluid carrying the cells or other biological material is directed to flow through this membrane, the cells or other biological material are retained inside the chamber by the membrane while the suspending fluid passes through and exits the chamber.

Since thin plastic membranes are usually quite flexible, a rigid porous material 110 is used as a backing. The material can be either a rigid porous plastic or a porous metal. Reagent fluids needed for cell and biological sample preparation are also passed through this membrane. Between the various cell treatment steps, the cells are washed by a mobile-phase fluid, which also enters the chamber through this port. However, following treatment, the stained cells are carried by the mobile phase fluid out of the chamber through exit port 108 to the flow cytometer for detection, counting and analysis.

The micro-chamber 100 is mounted on a solid piece of a heat conducting material 120 that is in thermal contact with a Peltier module 130 as illustrated in FIG. 1B When a DC electric current is passed through the Peltier module, heat is extracted from heat conducting material 120 causing it to cool. At the same time, heat rejected by the Peltier module is conducted to a heat sink 135 to permit heat dissipation to the ambient. A fan 150 can be operated to direct an air stream at the cooling fins, which are comprised of vertical slots cut into the solid heat conducting material 120 to increase the heat transfer surface area, thereby reducing the temperature difference across the Peltier module. The resulting power input to the Peltier module is reduced as well as the overall power consumption of the device.

When the DC voltage applied to the Peltier module is reversed in polarity, the electrical current flow is also reversed in direction. Heat is then extracted by the Peltier module from the ambient air through the heat sink and rejected to the conducting material 120 to cause it to be heated. As a result, the micro-chamber becomes heated. By this means, the micro-chamber can be maintained at a temperature either below or above the ambient to provide the optimal temperature environment for sample preparation and conditioning. This improves the accuracy and consistency of the overall process, leading to increased accuracy and consistency of cell preparation for flow cytometry. When cell activity is to be stopped almost completely for a given reaction, the temperature can be decreased to a sufficiently low level to cause a virtually complete freeze of the cell cycle. The Peltier module is provided with a temperature probe, and through automatic feedback control, the micro-chamber reactor 100 can be kept steadily at a specific temperature for a prolonged period of time if desired. The temperature can be increased or decreased quickly by using proportional-integral-differential (PID) control techniques that are well known in electronic and control theories.

Inside the micro-chamber 100 is a small magnetic stir bar 114. Mounted directly below the micro-chamber 100 is a small electric motor 140. The motor 140 has a rotating shaft 144 on which a small permanent magnet 142 is mounted. The permanent magnet establishes a magnetic field that penetrates into the interior volume of the micro-chamber. As the motor 140 rotates, this external magnetic field also rotates, causing the magnet 114 to rotate inside the micro-chamber. The rotating magnet 114 causes the fluid inside the chamber to be well stirred, producing a uniform cell suspension in the chamber on demand. This uniform cell suspension is then carried out of the chamber for detecting, counting or analyzing. Other mechanisms of providing a rotating magnetic field can also be used. These methods are well known to those skilled in the art of magnetic field and electric motor design, and will not be further described in this invention.

In some cases, it is desirable to use a microwave device allowing for microwave assisted cell preparation techniques to be used with the micro-chamber reactor. The microwave generator 160 is placed in proximity to the chamber, thus allowing the microwave energy to penetrate into the chamber. If the chamber walls are impermeable to microwave energy, the fluid containing the cell suspension can be circulated through plastic tubing that is permeable to microwave energy in a separate cavity in which the microwave energy is established for microwave assisted cell preparation. This and other methods of exposing cell samples to microwave energy are well known.

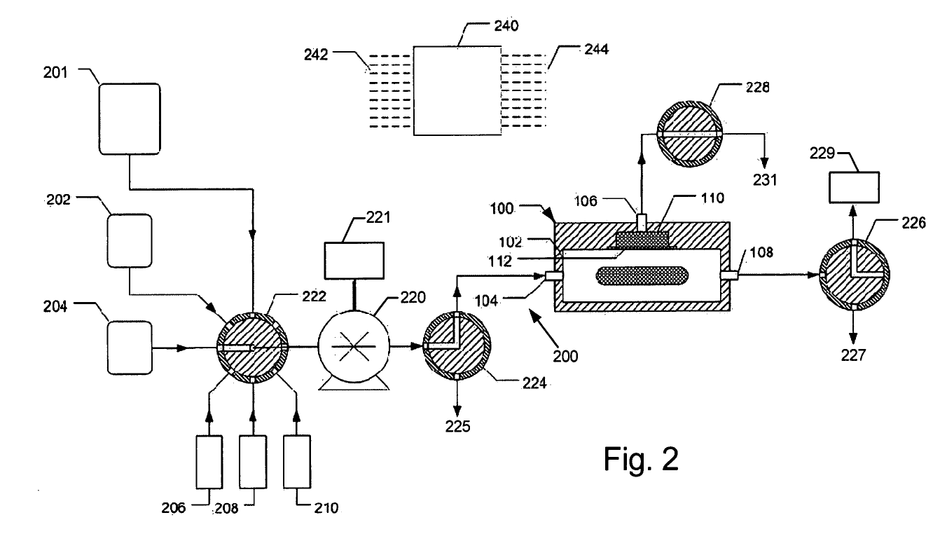

FIG. 2 shows a fluid transfer system generally indicated at 200 for the micro-chamber reactor to carry out the various cell preparation steps leading to cell staining and the transport and delivery of the stained cells in a mobile phase fluid to the flow cytometer. A positive displacement pump 220 with a stepper motor drive mechanism 221 is used for fluid pumping and transport. The speed of rotation of the stepper motor 221 can be varied by varying the frequency of electrical pulses applied to the stepper motor 221. The volume of fluid delivered can be accurately controlled by applying a known number of pulses to the stepper motor to cause it to rotate a specific number of steps and deliver a specific volume of fluid by the positive displacement pump 220. By this means, both the rate of fluid delivery and the volume of delivered fluid can be controlled electronically with accuracy and precision without using different pumps for different fluids being pumped.

The positive displacement pump 220 can be a conventional syringe pump with a stepper motor drive. It can also be a stepper-motor driven, multi-cylinder piston pump operated by a cam mechanism so that while one set of pistons are moving inside their cylinders to deliver fluid, the other set of cylinders are refilled with fluid from the pump intake. By this means, a continuous flow of fluid can be maintained from the inlet to the outlet. The synchronized piston motion can be provided mechanically by a cam, or electronically by synchronized electronic control of the piston motion in each cylinder. One such pump is the M6 pump made by Valco Instruments Company, Inc. of the U.S.

Fluids to be transferred through the micro-chamber reactor 100 are stored in their respective containers. These containers are fluidly connected to various inlet ports on the periphery of the multi-port valve 222. The central port of this multi-port rotary valve serves as the common outlet, which is connected to the inlet of pump 220.

The multi-port valve 222 has a rotor which, through an electronic actuator, can be rotated to a specific position to allow fluid connected to any of the inlet ports on the periphery of the valve to be connected to the single outlet port shown schematically at the center of the valve. By this means the fluid in any of the fluid reservoirs shown in FIG. 2 can be transferred by pump 220 to the micro-chamber reactor by simply adjusting the position of the rotor on this multi-port rotary valve.

Reservoir 201 is a source vessel for the biological cells. It can be a bioreactor containing the biological cells in a growth medium. In such cases, the reactor can be many liters, or even hundreds of liters in volume depending on the cells being grown and the specific application. Reservoir 201 can also be a small vessel with a volume of only a few milliliters (ml), into which a small volume of cell suspension can be injected manually for automated cell preparation following injection.

The cell suspension in reservoir 201 is pumped through the multi-port switching valve 222 by pump 220. The pump exit is connected to a 3-way valve 224, which can be positioned to direct the flow to enter the micro-chamber reactor 100 or go to waste 225. Outlet port 108 on micro-chamber 100 is connected to a valve 226, which has three positions: one being a shut-off position to cut off the flow completely, the other positions being used to switch the flow from the chamber to the flow cytometer 229 or to waste 227. At the shut-off position, the valve is blocked, and no fluid can pass through exit port 108. At this position, and with shut-off valve 228 downstream of exit port 106 being open, fluid entering the micro-chamber through valve 224 must pass through the membrane, through the exit port 106 and then through valve 228 to waste 231. Part or all of the waste fluid can also be directed into a high performance liquid chromatograph (HPLC) or other suitable instruments for chemical analysis of the cell medium before it goes to the waste reservoir. When cells are being transported from its source, the stepper motor can be operated at a low speed to avoid cell damage during sample transport.

A specific volume of fluid carrying the cells can be pumped into the micro-chamber by applying a specific number of electrical pulses to the stepper motor driven positive displacement pump. The retained cells can then be subjected to washing, fixing and staining, and all the steps needed to prepare a stained cell sample for counting by a flow cytometer. During these sample preparation steps, the two-way shut-off valve 228 downstream of port 106 would remain open, while valve 226 would remain closed. With valves in these positions, fluids can be pumped from their respective reservoirs into the micro-chamber for treatment. By this means, the cells are prepared and become ready for analysis by flow cytometry.

The specific sequence of steps involved in a typical sample preparation sequence is, briefly, as follows. During the step when a specific volume of cell suspension is introduced into the micro-chamber, valve 222 is in the position allowing the cell suspension to flow into the chamber. Valve 222 is then switched to the position to allow the mobile phase fluid in reservoir 204 to be pumped into the chamber. A widely used mobile phase fluid is phosphorous buffered saline (PBS). During this step, the growth medium in which the cells are suspended is replaced by the mobile phase fluid. The mobile phase fluid can be pumped at an appropriately high rate by the variable speed, stepper-motor driven pump 220 in order to complete the fluid displacement and cell washing step in a short time.

Following washing, the reagent for cell fixing can be pumped into the micro-chamber 100 to expose the cells to the fixing reagent. A commonly used fixing reagent is ethanol, which is contained in reagent reservoir 206. The multi-port rotary switching valve 222 can be switched to the position allowing the fixing reagent in reservoir 206 to be pumped into the chamber. Only three reagent reservoirs are shown in FIG. 2 for clarity. More reagent reservoirs can be provided depending on need and application.

After cell fixation, the rotary valve 222 is switched back to the position to allow the mobile phase fluid in reservoir 204 to flow into the micro-chamber 100 to displace the fixing solution in the micro-chamber 100 and wash the cells free of the fixing reagent. Following washing, the rotary valve 222 is switched to a position to allow the staining reagent in reservoir 208 to be pumped into the chamber. After allowing sufficient time for the stain-uptake process by the cells and staining reaction to complete, a process called incubation, the rotary valve is switched again to the position to allow the mobile-phase fluid in 204 to flow through the micro-chamber to thoroughly wash the cells free of the remaining staining fluid. Upon completing this final washing step, valve 228 will close and valve 226 will open to allow the fluid from the chamber carrying the suspended cells to flow into the flow cytometer for cell detection and counting.

Additional reagents can be provided. In some applications, the cells can be treated by an immuno-assay technique. In such a case one of the reagents would represent the antibody solution that can be injected into the micro-chamber for the appropriate immuno-reaction to take place. The number of reagents needed thus depends on the application. More reagent reservoirs can be provided when needed.

The operational control of the micro-chamber reactor as well as the recording of data generated by the optical cell detector described later is handled by an electronic controller 240. Because of the complex nature of the control and data processing functions, a micro-processor based computer 240 is generally used. The computer 240 with its analog and digital input signal lines 242 and analog and digital output signal lines 244 allow the computer 240 to receive the input signals from the sensors and provide output signals to control the system operation.

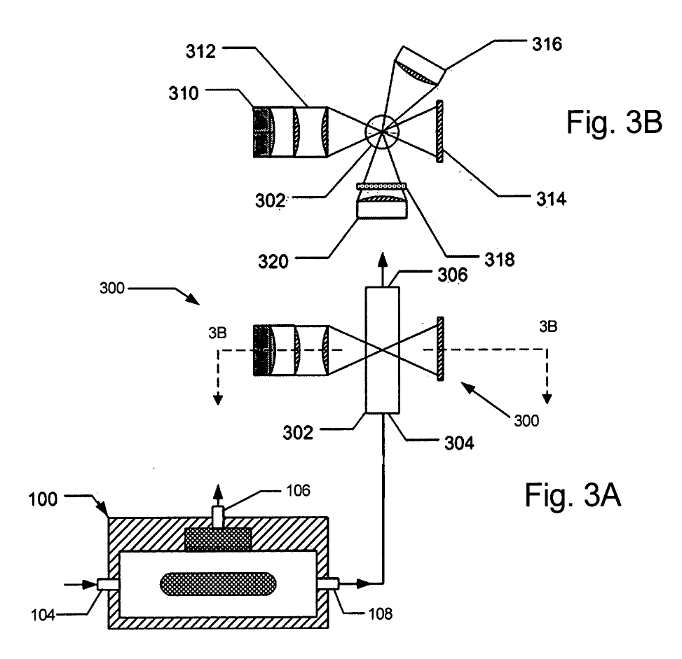

FIGS. 3A and 3B are schematic views showing the design of an optical detector 300 for detecting stained biological cells. The stained and treated cells from the micro-chamber reactor are directed to flow into a tube 302 in the detector. Tube 302 has an inlet 304 and an outlet 306. The tube 302 is transparent, and usually made of glass, quartz, or sapphire, to allow light from a laser light source 310 to pass through the lens system 312 and be focused in the fluid flow passageway inside tube 302. To facilitate light transmission through the fluid passageway, the tube cross-section may be rectangular. Other light sources, such as light from an electric discharge lamp can also be used. As the suspended cell particles, illuminated by the focused light, pass through the volume illuminated by the focused light in the tube 302, light is scattered by inelastic light scattering. This inelastically scattered light signal is collected by the lens in front of the photo-detector 316 and directed to fall onto the sensing surface in the detector to produce an output electrical signal in proportion to the amount of light detected by the sensing surface in sensor 316. The output signal is processed electronically by an amplifier, which usually forms an integral part of the sensing and amplification electronics of sensor 316. The amplified signal is in the form of an electrical pulse, with a pulse height that is proportional to the scattered light detected by sensor 316. These pulses are then subjected to electronic pulse height analysis by suitable pulse height analysis circuitry (not shown) for subsequent storage and processing by computer 240. In addition, a photo-detector with a filter 318 allows the laser induced fluorescent light signal from the stained cells to pass through and be detected by optical sensor 320. More than one such detector can be used to detect fluorescent light signals in several wavelength bands. For clarity only one such detector is shown. The output signal from the detector 320 is similarly amplified electronically and processed by pulse height analysis circuitry (also not shown) for subsequent recording and further processing by computer 240.

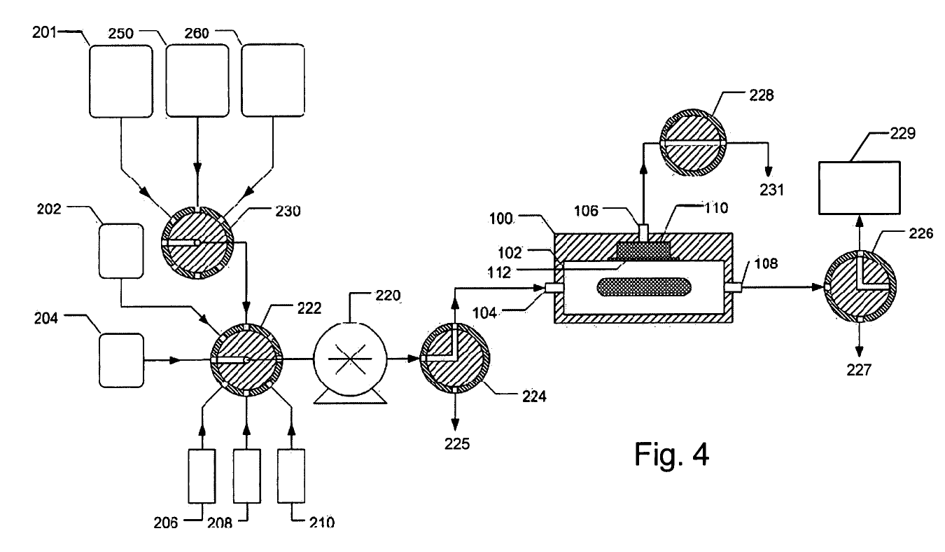

When more than one source of biological cells is to be prepared and stained in the micro-chamber reactor with the same fluid transfer system, the system in FIG. 4 can be used. For clarity, only three cell sources, 201, 250 and 260 are shown. In practice, more than three cell sources can be sampled by the same fluid transfer system. Each cell source, 201, 250 or 260 may be a bioreactor in which cells are grown in a growth medium. During cell growth, each bioreactor can be sampled periodically at predetermined intervals. The cells can then be processed automatically for detection and counting by a flow cytometer.

Sampling from each bioreactor can be made with a rotary valve 230 with the required number of fluid inlet ports to allow cell sample from each reactor to flow through to the common outlet port on the center. The fluid containing the cell sample then flows from this outlet port into the rotary valve 222 which is part of the fluid transfer system described earlier with respect to FIG. 2. Following sampling, the cells are then prepared and stained for delivery to an external flow cytometer or the included flow cytometric detector that forms part of the same automated sample preparation and detection system described in this invention. The process is then repeated for each bioreactor. All three reactors can be sampled at periodic, pre-determined intervals to allow the growth of cells in the reactors to be monitored. By this means, bioreactors in a large production facility can be automatically monitored with output data from the flow cytometer being delivered to a central computer for overall production system control and operation.

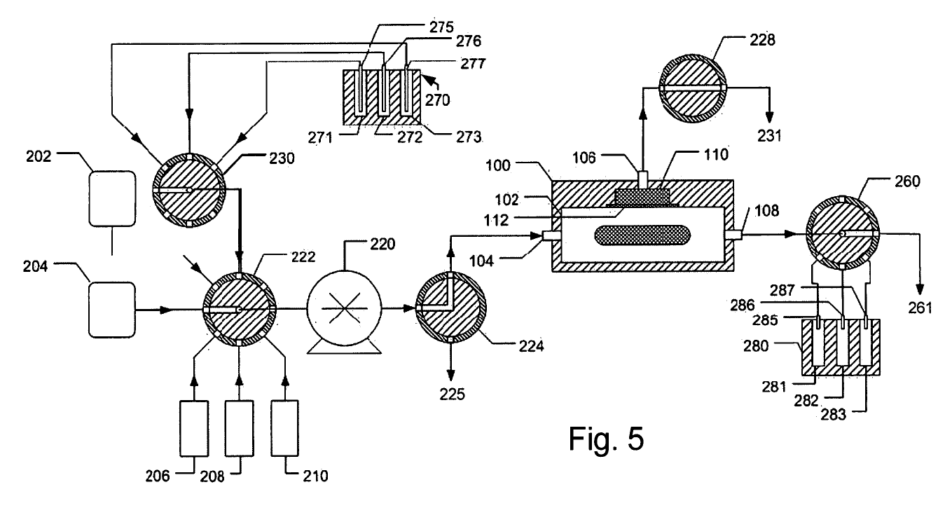

FIG. 5 shows a configuration allowing for manual sample introduction and removal with the sample preparation and staining system. The sample holder 270 is provided with one or more sample wells into which the samples can be manually introduced by a syringe or a pipette. Only three wells, 271, 272, and 273 are shown for clarity. Capillary tubes 275, 276 and 277 are used to allow sample in each well to be drawn into the system through rotary valve 230 for sample preparation and staining. Following staining, the stained samples are returned to sample holder 280 in which the wells 281, 282 and 283 are located. The treated samples are delivered to these wells through rotary valve 260, and the respective capillary delivery tubes 285, 286 and 287. The valve 266 also has a pathway to waste 261. By this means one or more cell samples can be introduced into the sample preparation and staining system and the prepared and stained samples can be transferred to a separate set of receiving wells automatically. Well holder 280 containing the stained samples can then be removed from the system and placed in a flow cytometer for subsequent sampling and measurement by the flow cytometer.

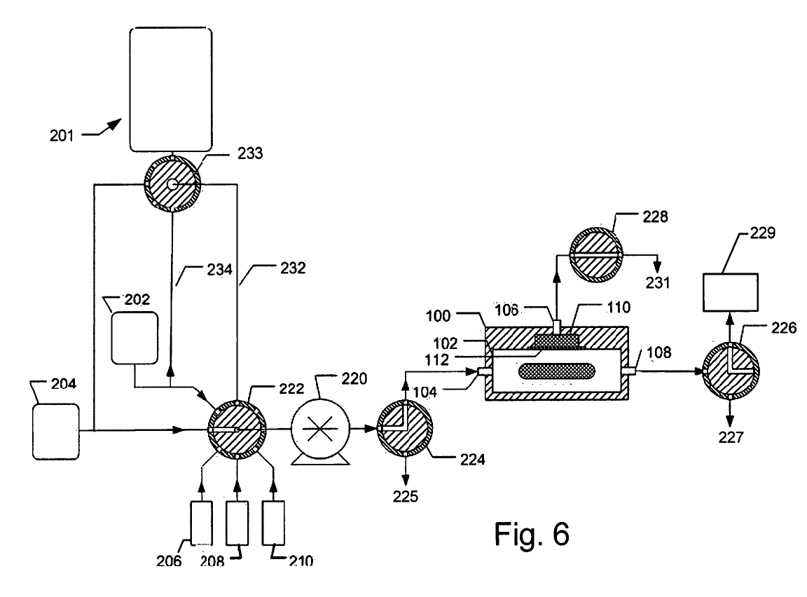

When sampling cells from a bioreactor that may be located some distance away from the cell preparation and staining system it is necessary to consider issues related to sample transport in long sampling lines. FIG. 6 is a schematic view of a sample transport and line cleaning and maintenance apparatus that can be used for automatic cell preparation and detection described in this invention.

Mounted on the bioreactor, or in close proximity to it, is a multi-port rotary valve 233 allowing three or more fluid sources to be connected to the periphery inlet ports on the valve. Fluid from each source can thus be directed to flow through the common outlet port on the center. During sampling, the stepper motor pump is operated at a moderately high speed to draw the sample through sampling line 232. The pump speed must not be so high as to cause cell damage during transport. Upon reaching the inlet of the pump 220, the sample flow can be reduced to allow the fluid to flow slowly through the pump in order to avoid cell damage during their passage through the pump chambers. Upon leaving the pump, the fluid then flows through the 3-way shut off valve 224 in the position shown leading to the micro-chamber reactor. Following sample extraction and subsequently sample preparation as described earlier, the cells are delivered to the internal flow cytometric detector or an external flow cytometer for detection. The pump can then be stopped and the valve position changed for cleaning the sampling line.

For cleaning and disinfecting sampling line 232, rotary valve 233 is switched to the position to allow the cleaning and disinfection fluid stored in reservoir 202 to flow through valve 233, then through valve 224 and into waste 225. Following line cleaning and disinfection, valve 233 is switched to allow the mobile phase fluid stored in reservoir 204 to flow through valve 233, then through sampling line 232, valve 222, pump 220, then through valve 224 to waste 225. The entire fluid passage can thus be rinsed by the mobile phase fluid. Following rinsing with the mobile phase fluid, the system can be placed on standby with valve 233 being switched to a blocked standby position without any fluid flow in the sampling lines, until another sample is to be taken and prepared as described above.

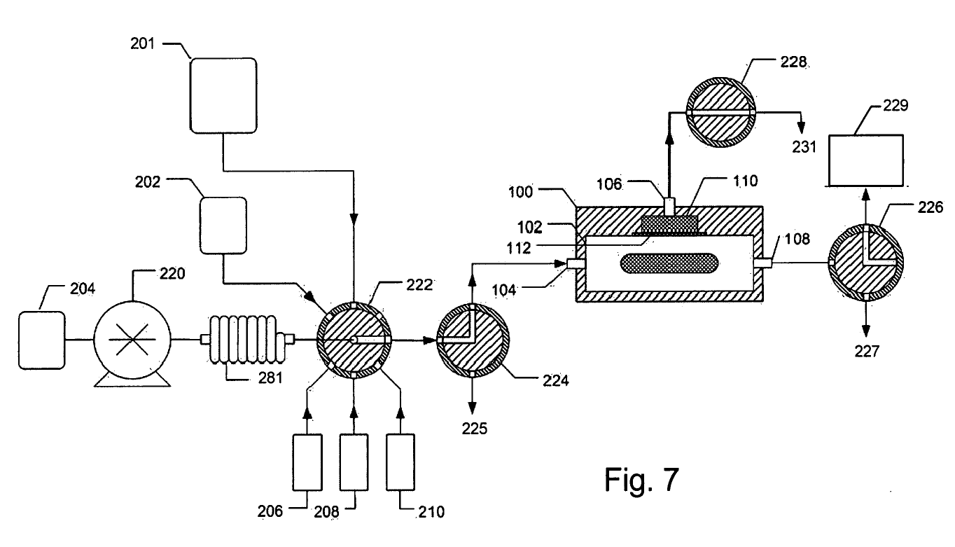

To avoid cell damage during sample transport, the fluid transfer system shown in FIG. 7 can be used. The system in FIG. 7 differs from those in FIGS. 2 - 6 in having the pump 220 located upstream, rather than downstream, of valve 222. In addition, a coil of tubing 281 is located between pump 220 and valve 222 to allow a sample to be drawn into the coil from the cell source 201. Pump 220 is a bidirectional pump that can be operated in a direction that creates suction and draws the sample from source 201 into the coiled tubing 281. The coiled tubing 281 is used as a repository for the sample. The internal volume of the coil tubing is larger than the sample volume being withdrawn. As a result only part of the volume of coil 281 is filled with the sample fluid from source 201.

During sample delivery the direction of the pump is reversed. The mobile phase fluid is pumped by pump 220 into the coiled tubing 281 to push the sample in the coiled tubing through valve 222 and into the micro-chamber. The cell sample would thus flow in and out of the coiled tubing 281 without actually passing through the pump chambers. Damage to the cells can thus be substantially eliminated. In comparison, the systems shown in FIGS. 2 - 6 require the sample fluid to flow through the pump chambers, where cells may suffer damage due the pumping motion of the positive displacement pump. Although the pumping speed can be reduced to minimize cell damage as previously explained, cell damage will likely occur and cannot be totally eliminated. In comparison, the system in FIG. 7 avoids the flow of sample fluid through the pump completely, thereby reducing or eliminating possible cell damage.

Following sample delivery to the micro-chamber, the delivery of other fluid reagents that are needed for fixation and staining can be accomplished in a similar manner. To deliver the fixative from reservoir 206, for instance, valve 222 is rotated to the position to allow the reagent to be drawn by pump 220 into the coiled tubing 281. After the required volume of the reagent is drawn into the coiled tubing 281, valve 222 is switched to allow the reagent in coil 281 to be connected through 3-way valve 224 to the micro-chamber. The rotational direction of pump 220 is then reversed to allow the mobile phase fluid to be pumped from its storage reservoir 204 into the coiled tubing 281 thereby displacing the reagent in the coiled tubing 281 and forcing it to flow through valve 222 and valve 224 into the micro-chamber. The delivery of other reagent fluid including the staining reagent can be accomplished in a similar manner and will not be further described.

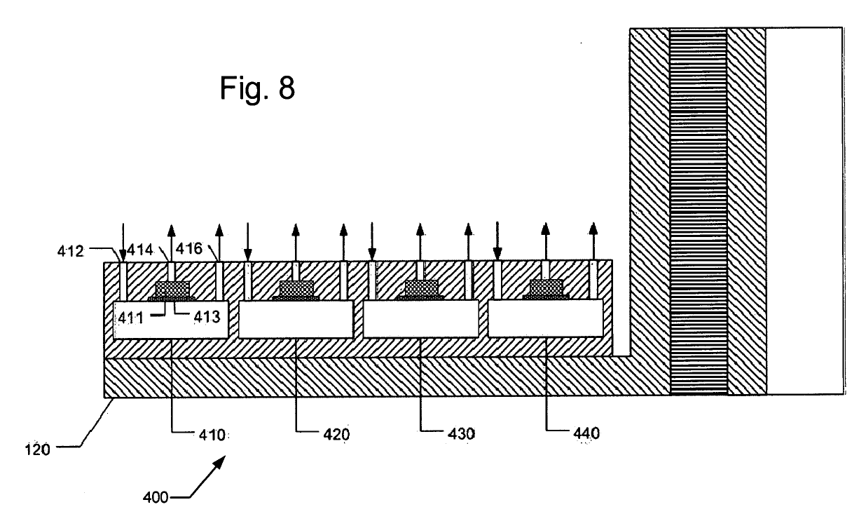

Another aspect of this invention relates to the use of a multi-chamber reactor generally indicated at 400 to process several biological cell samples in quick succession. FIG. 8 shows a multi-chamber reactor 400 fabricated from a single piece of plastic or metal in which the chambers are located. For clarity purposes, only four chambers are shown: 410, 420, 430 and 440. Each chamber is provided with an inlet port and two exit ports as previously described. Chamber 410 is a typical chamber with one inlet port 412 and two outlet ports 414 and 416. It also contains a membrane material 413 supported by a rigid, porous backing 411. This multi-chamber reactor 400 can be placed in thermal contact with a thermally conductive material that is in contact with a Peltier cooling and heating module as previously described. The same fluid transfer system can be used to draw cell samples into each chamber. Alternatively, several fluid transfer systems can be used to reduce the time needed for cell preparation.

Following cell sampling into each chamber, the chambers can be filled with a fluid which may be a culture medium for cell growth or a reagent fluid. Each cell sample can be subjected to a specific treatment by adding a reagent in the form of a drug or chemical compound of a specific amount to the cell sample to study its effect on cell expression. Following reagent injection, the cells can then be kept at a specific temperature in each chamber for a specific time period. This can then be followed by the sample preparation and detection steps previously described.

This multi-chamber reactor system 400 is particularly useful for laboratory research including research for drug development. For such applications, large number of chambers may be needed. For the purpose of this invention, the number of chambers may be arranged from as few as two, to as many as may be needed or deemed appropriate for a given application. The total number of chambers used can be large or small without violating the fundamental principles described in multi-chamber approach to cell processing as described herein.

The sample preparation and staining aspect of the present invention is particularly useful for preparing cell samples for measurement by a stand-alone flow cytometer. Such flow cytometers are frequently equipped with a multi-well sampling plate. A commonly used multi-well sample plate is a 96 well plate in which 96 individual wells are provided to store small volumes of fluid, typically a fraction of a milliliter each in the individual wells. The wells are typically arranged in 12 rows or columns of 8 wells each. Each well is usually filled with one cell sample that needs to be treated, prepared, and stained for measurement by the flow cytometer.

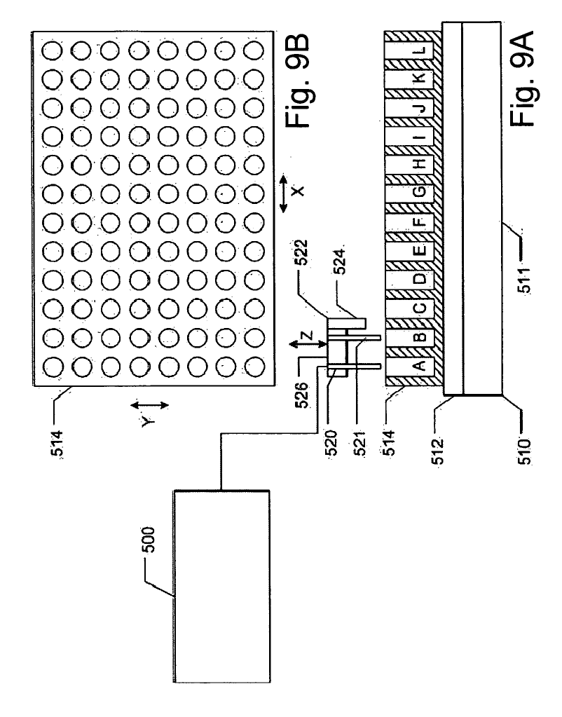

FIGS. 9A and 9B show a multi-well sample handling system generally indicated at 501 combined with the automatic sample preparation system described in this invention, generally indicated at 500. The automatic sample handling system includes an XY stage 510 on which a multi-well sample plate 514 is mounted. In FIGS. 9A and 9B, a 96 well sample plate is shown for clarity and for purposes of illustration. In practice, almost any number of wells can be used. This 96 well sample plate is mounted on a stepper-motor controlled XY stage 510 comprised of a fixed base 511 and a movable stage 512. Two capillary tubes 520 and 521 are mounted on a Z-stage 522. The Z stage is comprised of a fixed base 524 and a movable stage 526 to which the capillary tubes 520 and 521 are attached. The Z-stage is moved up to allow the XY stage below to move freely in the XY direction. Upon reaching a desired XY position, the Z-stage is lowered to move the sample capillary into their respective wells. One tube 520 is for sample intake, while the other 521 is for sample delivery. In the specific location shown in FIG. 9A, capillary 520 is positioned to be lowered into the sample well in column A allowing the sample in the well to be taken up into the sample preparation system. The second capillary 521 is position to be lowered into sample well in column B so that the stained sample can be delivered into the well in column B. By this means samples stored in alternate columns A, C, E, etc. are prepared and delivered into wells in columns B, D, F, etc.

The 96 well sample plates are often used for screening applications. In such applications, cells can represent different species or mutant cell lines, and the purpose of the flow cytometry analysis is to identify a specific cell type that has specific characteristics. Another type of screening application uses the same test cells and requires identification of a cellular reaction towards a substance that the cells are exposed to. In both types of screening applications the cells are screened by flow cytometry, and the cells must be prepared for flow cytometric measurement.

When using the sample handling and preparation system shown in FIGS. 9A and 9B for cell screening in drug development, the cell sample is first withdrawn from a cell source or several sources. After a specific chemical or drug compound is added to a sample, the sample is delivered into a well. Usually only half of the wells would be filled with samples so treated and half would be left empty to receive samples that have already been prepared for flow cytometry. The automatic sampling preparation and treatment system can be used both for sample treatment, and for subsequent sample preparation and staining for flow cytometry. Following cell treatment and sample preparation and staining, the entire 96-well plate can be removed from the system and loaded into a flow cytometer similarly equipped with an automated 96-well sample handling system to allow the stained sample to be drawn into the flow cytometer for measurement. The loading and unloading of the 96 well plates can be done manually, or by means of a robot for fully automated operating of the screening process.

Although the present invention has been described with reference to preferred embodiments, workers skilled in the art will recognize that changes may be made in form and detail without departing from the spirit and scope of the invention.

What is claimed is:

1. A system to identify or screen one or more biological materials comprising:
at least one bioreactor adapted to provide biological samples containing cells, cell components, cell-derived materials or a combination thereof in a fluid;
an automated sample handler comprising at least one bi-directional pump to transport biological samples, reagents and fluids, at least one reaction chamber with at least two regions separated by a semi-permeable membrane and fitted with a thermoelectric module for cooling and heating the chamber, and at least one multi-positional valve to direct transport of the biological samples, reagents and fluids into and out of the reaction chamber, and a microprocessor to control operations of the sample handler;
a detector to identify biological material in the samples.

2. The system according to claim 1 having two or more bioreactors.

3. The system according to claim 1 further comprising including a sample coil located downstream of the pump and upstream of the reaction chamber.

4. The system according to claim 1 having two or more reaction chambers.

5. The system according to claim 1 wherein the detector comprises a flow cytometer.

6. The system according to claim 1 wherein the sample repository comprises a multi-well plate.

7. An apparatus for preparing a biological sample for detection, the apparatus comprising:
a chamber with a semi-permeable membrane adapt for receiving biological material in a fluid;
a mechanism to transfer the biological material including a pump;
a valving mechanism in cooperation with the mechanism to transfer the biological material for directing flow of the biological material into the chamber;
a mechanism for treating the biological material in the chamber including one of:
a thermoelectric module for cooling and heating the chamber; or
a microwave mechanism to irradiate the biological sample within the chamber;

8. The apparatus of claim 7 and further including a repository for accepting biological material from the chamber.

9. The apparatus of claim 7 with at least two chambers each chamber being fitted with a semi-permeable membrane

10. The apparatus of claim 8 wherein the repository is a multi-well plate.

11. An apparatus for preparing a biological sample for detection, the apparatus comprising:
a chamber with a semi-permeable membrane adapted for receiving a biological material in a fluid;
a mechanism to transfer the biological material including a pump, the pump being on of the following types:
a multi-cylinder piston pump;
a multi-cylinder piston pump operated by a stepped motor; or
a bi-directional, multi-cylinder piston pump; and
a valving mechanism in cooperation with the mechanism to transfer the biological material.

12. The apparatus of claim 11 and further comprising a sample coil positioned downstream of the pump and upstream of the chamber.

13. The apparatus of claim 11 and further including two or more chambers.

14. The apparatus of claim 11 and further including a magnetic stirrer within the chamber, the stirrer being propelled by a magnetic stirring mechanism.

15. An apparatus for preparing a biological material for subsequent processing by a detection mechanism, the apparatus comprising:
a housing having an inlet and an outlet, said housing including
a biological material preparing chamber adapted for receiving a biological material in a fluid;
a mechanism to provide motive force to transport the biological material into the inlet;
a biological material preparation mechanism that prepares the biological material in the chamber; and
a biological material delivery mechanism that transports the biological material through the outlet.

16. The apparatus of claim 15 and further including an input receptacle for providing a biological material and in fluid communication with the inlet such that the biological material is transportable through the inlet and an output receptacle for storing the prepared biological material, the output receptacle being in fluid communication with the outlet.

17. The apparatus of claim 15 and further including a plurality of input receptacles and a plurality of output receptacles and an input valving mechanism for providing fluid transport passages such that biological material can be drawn from selected input receptacles and an outlet valving mechanism for providing passages to transport the prepared biological material to selected output receptacles.

18. The apparatus of claim 15 and further including a well plate having a plurality of wells, a portion of which include biological material for transport through the inlet, and a portion of which are for storing prepared biological material received through the outlet.

19. The apparatus of claim 18 and further including a platform operated by stepper motors and being moveable horizontally in two perpendicular directions such that a portion of the plurality of wells are positioned in fluid communication with the inlet for drawing biological material.

20. The apparatus of claim 18 and further including a platform operated by stepper motors on which the well plate is disposed , the platform being moveable in two directions perpendicular to each other such that a portion of the wells are positionable by movement of the platform to a position for receiving prepared biological material.

21. The apparatus of claim 15 and further including:
a biological material source in which the biological material is developed and in transport communication with the inlet for providing biological material to the inlet; and
a flow cytometer in fluid communication with the outlet for receiving and detecting prepared biological material from the outlet.

22. A biological sample preparation and detection apparatus for preparing and detecting biological material within a biological sample, the apparatus comprising:
a housing having a chamber for receiving the biological sample and an inlet in fluid communication with the chamber;
a transport mechanism for transporting the biological sample through the inlet;
a processing mechanism to prepare the biological sample for subsequent detection of selected biological material;
a detection mechanism for detecting the selected biological material from the prepared biological sample; and
a thermoelectric cooler and heater in heat transfer relationship with the chamber for providing a selected temperature environment within the chamber.

23. The apparatus of claim 22 wherein the detection mechanism comprises:
a light source capable of inducing fluorescent light emission from the biological material;
a first photo detector; and
a first filter positioned to permit fluorescent light to reach the photo detector.

24. The apparatus of claim 23 further including at least one additional photo detector and at least one additional filter to detect fluorescent light in a different wave length range than the first photo detector and the first filter.

25. The apparatus of claim 22 and further including:
a biological material source for developing biological material, the source being in fluid communication with the inlet; and
a valving mechanism to produce a fluid passage for transport of biological material to the inlet and to produce fluid passages for cleaning fluid to flow and clean passages between the biological material source and the inlet.

26. A process to identify or screen a biological material in a fluid sample comprising:
removing a fluid sample from a cell suspension by pulling the fluid sample into a sample loop with a bidirectional pump that is downstream from the sample loop;
transporting the sample in the sample loop to a reaction chamber having at least two regions separated by a semi-permeable membrane by pushing the sample from the sample loop to the reaction chamber with the bidirectional pump;
contacting the sample in the reaction chamber with at least one reagent to provide a detectable biological material;
transporting the detectable biological material to a detector;
identifying the detectable biological material with the detector to provide an identified biological material; and
transporting the identified biological material to a repository.

27. The process of claim 26 wherein the sample is processed by filtering, washing, fixing, staining, incubating or diluting the sample before contacting the sample with at least one reagent to provide the detectable biological material.

28. The process of claim 26 wherein the sample is heated or cooled or both heated and cooled during the contacting step with at least one reagent in the reaction chamber.

29. The process of claim 26 wherein the sample is contacted with more than one reagent.

30. The process of claim 26 wherein the reagent is a fixing reagent.

31. The process of claim 26 wherein the reagent is an immunoassay reagent.

32. The process of claim 26 wherein the identified biological material is a cell or cellular component.

33. The process of claim 26 wherein the identified biological material is a peptide or protein.

34. The process of claim 26 wherein the detector is a flow cytometer.

35. The process of claim 26 wherein the repository is a multi-well plate

36. The process according to claim 26 wherein one or more steps are automated.

37. The process of claim 26 wherein one or more steps are microprocessor controlled.

38. The process of claim 26 wherein the bidirectional pump is a stepper-motor driven, variable speed, multi-cylinder piston pump.

39. A method for transporting a sample of biological material from a first location to a second location through a fluid passage, the method comprising:
providing a valving mechanism between the first location and the second location, the valving mechanism having a plurality of ports to effect different fluid passages;
providing a repository in fluid communication with one port of the valve;
providing a pump in fluid communication with the repository;
operating a pump in a suction mode after the valve produces a first passage between the first location and the repository such that the sample of biological material is temporarily positioned within the repository; and
operating the pump in a pumping mode after the valve produces a second passage between the repository and the second location such that the sample of biological material is transported from the repository to the second location.

40. An apparatus to transport a sample of biological material, the apparatus comprising:
a source of biological material;
a destination for receiving the biological material;
a valving mechanism having a plurality of ports to effect different fluid passages, the valve being in fluid communication between the source and the destination;
a repository for temporarily receiving a sample of the biological material;
a pump in fluid communication with the repository, the pump having the capability of acting on the repository in suction mode and in pumping mode such that when the valve is operated to produce a first passage between the source and the receptacle, the pump is operational in a suction mode transporting a sample of the biological material into the repository, and when the valving mechanism is in a second position producing a passage between the repository and the destination, the pump is operational in a pumping mode to transport the biological sample to the destination.
